(19) United States
(12) Patent Application Publication
(10) Pub. No.: US 2025/0312017 A1
(43) Pub. Date: Oct. 9, 2025

(54) SENSOR UNIT WITH CONDUCTIVE THREADS FOR CHARACTERIZING A BIOLOGICAL LIQUID BASED ON DRYING BEHAVIOUR

(71) Applicant: FIBRA INC., Vaughan (CA)

(72) Inventors: Parnian MAJD, Vaughan (CA); Milad GHALAMBORAN, Aurora (CA)

(21) Appl. No.: 19/173,264

(22) Filed: Apr. 8, 2025

Related U.S. Application Data
(60) Provisional application No. 63/631,762, filed on Apr. 9, 2024.

(51) Int. Cl. A61B 10/00

(52) U.S. Cl. CPC A61B 10/0012; A61B 2010/0016; A61B 2560/0462

(57) ABSTRACT

A sensor unit for characterizing a biological liquid includes a plurality of conductive threads incorporated into a textile and a microcontroller electrically connected to the threads. The microcontroller is configured to apply a test signal to a first conductive thread and receive feedback signals from a second conductive thread. The feedback signals indicate that a biological liquid is electrically connecting the conductive threads. By comparing the feedback signals received at two or more time points, the microcontroller can compute a drying metric for the biological liquid. Additional features include monitoring the change in voltage of the feedback signal over time and estimating the volume of the liquid. The sensor unit may be incorporated into a wearable garment or included in a fertility monitoring system. In the fertility monitoring system, a computing device receives and compares the feedback signals to determine the drying metric for the biological liquid.

### FIELD

The present specification is directed to wearable devices, and in particular, a sensor for characterizing biological liquids based on drying behaviour.

### BACKGROUND

Smart clothing including moisture monitor systems are designed for diapers or similar sanitary products. These systems may incorporate electrodes that are positioned on an electrically insulating material in order to detect changes in the potential difference between the electrodes. The primary function of such devices is to monitor and alert for the presence of moisture, indicating leakage or wetness events.

### SUMMARY

The present specification improves upon conventional moisture monitoring systems by providing a sensor unit for a wearable device configured to evaluate the drying behaviour of a biological liquid. By monitoring the drying time or rate, the sensor unit enables characterization of the liquid, in contrast to prior solutions that merely detect the presence of moisture.

An aspect of the specification provides a sensor unit for characterizing biological liquids. The sensor unit includes a plurality of conductive threads incorporated into a textile, including at least a first conductive thread and a second conductive thread spaced from the first conductive thread. A microcontroller is electrically connected to the plurality of conductive threads. The microcontroller is configured to apply a test signal to the first conductive thread at a plurality of times, including a first and second time. The microcontroller is further configured to record a plurality of feedback signals in at least the second conductive thread responsive to a biological liquid electrically connecting the first and second conductive threads. The plurality of feedback signals includes at least a first and second feedback signal, corresponding to the first and second times, respectively. The microcontroller is further configured to compare the plurality of feedback signals and determine a drying metric for the biological liquid based on the comparison.

In one example, the microcontroller is further configured to detect whether the first feedback signal is transmitted by the second conductive thread and, if not transmitted, determine that no biological liquid is present on the textile.

In one example, the microcontroller is further configured to retrieve from memory a gap distance between the first and second conductive threads and determine the drying metric for the biological liquid based on the gap distance.

In one example, the microcontroller is further configured to determine a number of the conductive threads from which the first and second feedback signals are recorded, and the comparison of feedback signals includes comparing the respective number of conductive threads from which the first and second feedback signals are recorded.

In one example, the microcontroller is further configured to measure the voltage of the first and second feedback signals, and the comparison includes determining a difference between the voltage of the first and second feedback signals.

In one example, the microcontroller is further configured to record the plurality of times, and the comparison includes determining a time difference between the first and second times.

In one example, the microcontroller is further configured to record an end time when the second conductive thread ceases to transmit the feedback signals, and the comparison includes computing a feedback signal duration based on the first time and the end time.

In one example, the microcontroller is further configured to measure the voltage of the feedback signals, and the comparison includes computing a rate of change in the voltage.

In one example, the plurality of conductive threads is arranged in a grid pattern on the textile. The plurality of conductive threads comprises a first set of parallel threads and a second set of parallel threads perpendicular to the first set. The microcontroller is further configured to apply the test signal to a first one of the first set of parallel threads and a first one of the second set of parallel threads.

A further aspect of the specification provides a wearable device comprising a garment that includes a textile configured to be worn by a user and the sensor unit as described above.

A further aspect of the specification provides a fertility monitoring system comprising a sensor unit for detecting a biological liquid and a computing device. The sensor unit includes a plurality of conductive threads incorporated into a textile, including at least a first conductive thread and a second conductive thread spaced from the first conductive thread. A microcontroller is electrically connected to the plurality of conductive threads. The microcontroller is configured to apply a test signal to the first conductive thread at a plurality of times, including a first and second time. The microcontroller is further configured to record a plurality of feedback signals in at least the second conductive thread responsive to the biological liquid electrically connecting the first and second conductive threads. The plurality of feedback signals includes at least a first and second feedback signal, corresponding to the first and second times, respectively. The microcontroller is further configured to transmit the plurality of feedback signals. The computing device is configured to receive the plurality of feedback signals from the sensor unit, compare the plurality of feedback signals, and determine a drying metric for the biological liquid based on the comparison.

In one example, the microcontroller is further configured to detect whether the first feedback signal is transmitted by the second conductive thread and, if not transmitted, determine that no biological liquid is present on the textile.

In one example, the computing device is further configured to retrieve from memory a gap distance between the first and second conductive threads and determine the drying metric based on the gap distance.

In one example, the microcontroller is further configured to determine a number of the conductive threads from which the first and second feedback signals are recorded and transmit the respective number of conductive threads to the computing device. The comparison includes comparing the respective number of conductive threads.

In one example, the microcontroller is further configured to measure the voltage of the first and second feedback signals and transmit the respective voltages to the computing device. The comparison includes determining a difference between the respective voltages.

In one example, the microcontroller is further configured to record the first and second times and transmit the first and second times to the computing device. The comparison includes determining a time difference between the first and second times.

In one example, the microcontroller is further configured to record an end time when the microcontroller ceases to receive the feedback signal and transmit the end time to the computing device. The comparison includes computing a feedback signal duration based on the first time and the end time.

In one example, the microcontroller is further configured to measure the voltage of the corresponding feedback signals and transmit the voltage of the feedback signals to the computing device. The comparison includes computing a rate of change in the voltage.

In one example, the plurality of conductive threads is arranged in a grid pattern on the textile. The plurality of conductive threads comprises a first set of parallel threads and a second set of parallel threads perpendicular to the first set. The microcontroller is further configured to apply the test signal to a first one of the first set of parallel threads and a first one of the second set of parallel threads.

In one example, the computing device is further configured to compare the drying metric of the biological liquid to reference data and determine a reproductive status of a user based on the comparison between the drying metric and the reference data.

These together with other aspects and advantages which will be subsequently apparent, reside in the details of construction and operation as more fully hereinafter described and claimed, reference being had to the accompanying drawings forming a part hereof, wherein like numerals refer to like parts throughout.

### BRIEF DESCRIPTION OF THE DRAWINGS

Embodiments are described with reference to the following figures.

FIG. 1 is a front elevation view of a wearable device with a sensor unit, according to one embodiment.

FIG. 2 is a block diagram of the sensor unit of FIG. 1, according to one embodiment.

FIG. 3 is a block diagram of a fertility monitoring system including the sensor unit of FIG. 1.

FIG. 4A is a top elevation view of a sensing element for the sensor unit of FIG. 1, according to one embodiment.

FIG. 4B is a top elevation view of another sensing element for the sensor unit of FIG. 1, according to another embodiment.

FIG. 5A is a top elevation view of another sensing element for the sensor unit of FIG. 1, according to a further embodiment.

FIG. 5B is a top elevation view of a further sensing element for the sensor unit of FIG. 1, according to a yet further embodiment.

FIG. 6 is a block diagram of a method for characterizing a biological liquid using the sensing element of FIG. 4A, according to one embodiment.

FIG. 7A is a top elevation view of the sensing element of FIG. 4A, according to one embodiment.

FIG. 7B is a top elevation view of the sensing element of FIG. 4A, according to one embodiment.

FIG. 8 is a graph showing the relationship between voltage and time, according to one implementation of the sensor unit of FIG. 1.

### DETAILED DESCRIPTION

The present specification provides a sensing unit for evaluating the drying behaviour of a biological liquid. In the embodiments described herein, the sensing unit is adapted for use in a wearable device, however the sensor is not particularly limited and may be applied to any suitable textile.

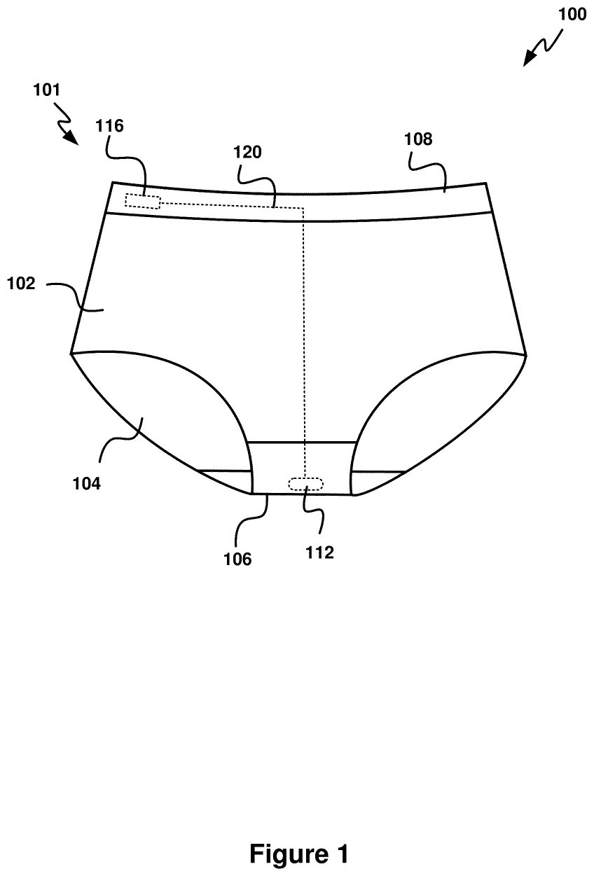

FIG. 1 is a front elevation view of a wearable device 100 including a sensor unit 101 according to one embodiment. In the example shown in FIG. 1, the wearable device 100 comprises underwear, however the wearable device 100 is not particularly limited. In other embodiments, the wearable device 100 comprises an undershirt, bra, chest strap, headpiece, leggings, swimwear, shapewear, shirt, sock, wristband, adhesive patch, or the like. The wearable device 100 generally comprises one or more textile portions to be worn on the user's body. In this example, the textile portions comprise a front portion 102, a rear portion 104, a gusset 106 and a waistband 108, however other configurations are contemplated. One or more of the textile portions may comprise a plurality of textile layers.

The textile portions may comprise any suitable woven or non-woven fabric. In examples where the textile portions comprise a woven fabric, the textile may include but is not limited to cotton, silk, linen, wool, polyester, nylon, rayon, modal, and a combination thereof. The textile may be selected to optimize the distribution and drying time of liquids contacting the textile. The drying time for absorbent fabrics like cotton is generally faster than the drying time for non-absorbent fabrics like nylon. The distribution of liquids is generally better on absorbent fabrics as opposed to non-absorbent fabrics. In some examples, the textile is selected to achieve a distribution time of about 5 to 10 seconds. In specific non-limiting examples, the textile comprises a fabric blend of cotton and polyester, and in particular examples about 10% polyester and about 90% cotton.

The wearable device 100 further comprises the sensor unit 101 for characterizing a biological liquid. The sensor unit 101 includes at least one sensing element 112 and a microcontroller 116 for receiving data from the sensing element 112 via a connector 120. The sensing element 112 may be incorporated into one of the textile portions by sewing, weaving, knitting, adhesion, or any other suitable method of incorporation. In the example shown in FIG. 1, the sensing element 112 is incorporated into the gusset 106, however the sensing element 112 is not particularly limited. In other embodiments, the sensing element 112 is incorporated into the rear, front, or waistband of the wearable device 100. Generally, the sensing element 112 is positioned to capture one or more biological liquids of interest secreted by the user.

The microcontroller 116 is configured to apply a test signal to the sensing element 112 and receive a feedback signal indicative of a characteristic of the biological liquid. The microcontroller 116 is configured to transmit the test signal to the sensing element 112 via the connector 120. The sensing element 112 is configured to transmit the feedback signal to the microcontroller 116 via the connector 120.

The connector 120 electrically connects the sensing element 112 to the microcontroller 116. The connector 120 may be disposed between two layers of textile, disposed on the surface of a layer of textile, knitted into the textile, stitched into the textile, or woven into the textile of the wearable device 100. In specific embodiments, the connector 120 comprises a conductive thread that is stitched into the textile portion of the wearable device 100. The connector 120 may comprise any suitable conductive material such as stainless steel. A coating may cover the connector 120 to protect the connector from oxidization.

The microcontroller 116 is preferably located in the waistband 108 of the wearable device 100 but the microcontroller 116 is not particularly limited. The microcontroller 116 applies a test signal to the sensing element 112 and receives a feedback signal responsive to the test signal.

In some examples, the wearable device 100 does not include a microcontroller 116 and instead includes a wireless transmitter for transmitting the feedback signal wirelessly. Suitable examples of wireless transmitters may include a Wi-Fi module, a Bluetooth™ module, a radio frequency identification (RFID) tag, the like, or a combination thereof.

In specific, non-limiting embodiments, the microcontroller 116 includes the Arduino™ UNO (Arduino: New York, United States) or the Arduino™ Nano 33 BLE (Arduino: New York, United States), however the microcontroller 116 is not particularly limited.

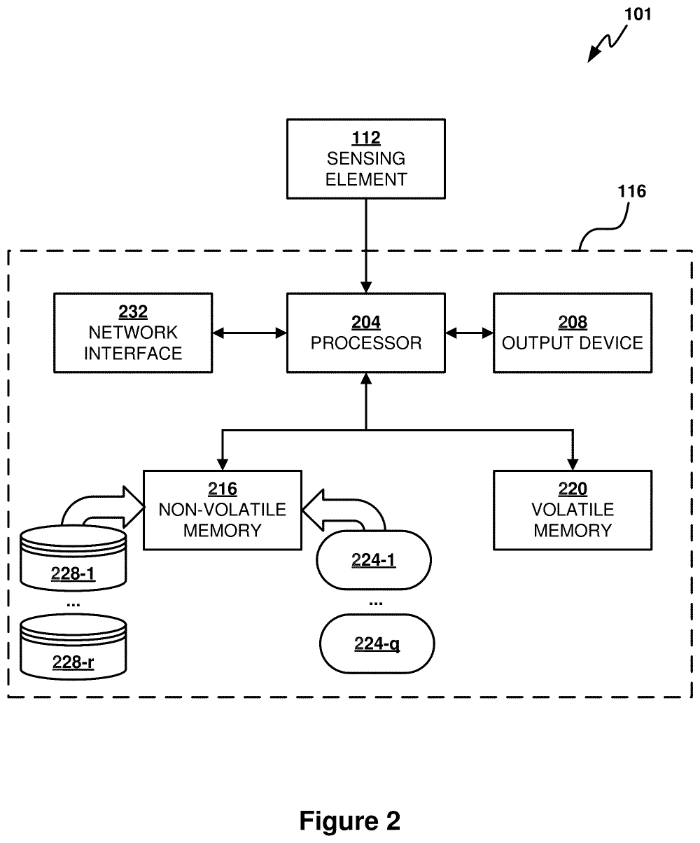

FIG. 2 in a block diagram of the sensor unit 101 showing the microcontroller 116 in greater detail. The microcontroller 116 may comprise a processor 204 for receiving a feedback signal from sensing element 112 and processing said feedback signal to generate an output.

The processor 204 may be implemented as a plurality of processors or one or more multi-core processors. The processor 204 may be configured to execute different programing instructions responsive to the feedback signal received via the sensing element 112 and to control one or more output devices 208 to generate output on those devices.

To fulfill its programming functions, the processor 204 is configured to communicate with one or more memory units, including non-volatile memory 216 and volatile memory 220. The non-volatile memory 216 can be based on any persistent memory technology, such as an Erasable Electronic Programmable Read Only Memory (“EEPROM”), flash memory, solid-state hard disk (SSD), other type of hard-disk, or combinations of them. The non-volatile memory 216 may also be described as a non-transitory computer readable media. Also, more than one type of non-volatile memory may be provided.

The volatile memory 220 is based on any random-access memory (RAM) technology. In specific, non-limiting examples, the volatile memory 220 can be based on a Double Data Rate (DDR) Synchronous Dynamic Random-Access Memory (SDRAM). Other types of volatile memory are contemplated.

The processor 204 also connects to a network via a network interface 232. Suitable examples of network interfaces may include a Wi-Fi module, a Bluetooth™ module, a radio frequency identification (RFID) tag, the like, or a combination thereof.

Programming instructions in the form of applications 224 are typically maintained, persistently, in non-volatile memory 216 and used by the processor 204 which reads from and writes to volatile memory 220 during the execution of applications 224. Various methods discussed herein can be coded as one or more applications 224. (Generically referred to herein as “application 224” or collectively as “applications 224” This nomenclature is used elsewhere herein.)

One or more tables or databases 228 are maintained in non-volatile memory 516 for use by applications 224.

The wearable device 100 further includes a power source (not shown) for powering the sensing element 112 and the microcontroller 116. The power source may be integrated with or connected to the microcontroller 116. The power source may include one or more batteries, power ports, self-charging power packs, a power generation unit, the like, or a combination thereof.

In examples where the power source includes a battery, the battery may be a rechargeable or non-rechargeable battery. The battery may be removable or non-removable from the microcontroller 116. The battery may be located in the waistband 108 with the microcontroller 116 or configured to be worn on the wrist of the user. In embodiments where the battery is adapted to be worn on the user's wrist, the battery may be integrated into a wristband. The battery is electrically connected to the microcontroller 116 for powering the microcontroller 116 and sensing element 112. In some examples, the battery is removably coupled to the wristband to allow for replacement or recharging independently of the wristband enclosure.

In specific non-limiting embodiments, the power source comprises one or more lithium-ion batteries. In these examples, the power source may be connected to a breadboard for transferring power to the microcontroller 116 and sensing element 112.

In specific, non-limiting embodiments, the power source includes the Panasonic™ Lumix Li-Ion Battery Pack (model no. DMW-BLF19). The 7.2V, 1860 mAh battery potentially works for up to 24 hours if the operating voltage of the microcontroller 116 is between 7 to 14 V. Other power sources such as fully self-charging power packs (FSPP).

In further non-limiting embodiments, the power source includes the Molex™ Thin-Film Battery (Mouser Electronics: Kitchener, Canada). the Molex™ Thin-Film Battery may be used to power the microcontroller 116 and the sensing element 112. The Molex™ battery has a shelf life of about two years and can operate in a humidity of about 20% to about 90% and in a temperature range of about −35° C. to about 50° C. It is a 3V battery with an initial internal resistance of about 90 ohms and a peak current (maximum) of about 8 to about 10 mA. It is bendable and small. It has a minimum bending radius of about 35.00 mm, a thickness of about 0.70 mm, and a width of about 36.00 mm.

In embodiments where the power source includes a power port for receiving power from an external source, the power source may further include a battery, and the power port may be configured to charge said battery. In a specific, non-limiting embodiment, the battery is charged via a serial USB port of an external computing device.

In embodiments where the power source includes a power generation unit, the power source may comprise a thermoelectric generator, a solar cell, piezoelectric device, an electromagnetic generator, the like, or combinations thereof.

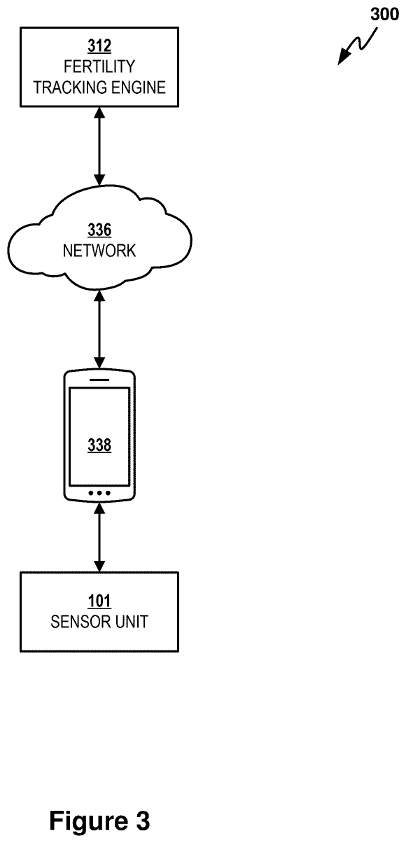

The network interface 232 can be used to connect a computing device, thereby obviating the need for one of more components of the microcontroller 116. FIG. 3 shows a fertility monitoring system 300 according to one embodiment in which the sensor unit 101 connects to a computing device 338.

The computing device 338 can be any type of human-machine interface for interacting with the sensor unit 101. For example, the computing device 338 may include a smartphone, a personal computer, a tablet computer, a smartwatch, a smart home system, or any other device that can be used to receive and send content. The computing device 338 can be operated by a user associated with a respective identifier that uniquely identifies the user accessing the computing device 338. The computing device 338 may comprise a processor for executing programming instructions in the form of applications. The computing device 238 may further include non-volatile memory. The computing device 238 may further include volatile memory. The computing device 338 may further include an output device. Any description of the processor 204 may apply to the processor of the computing device 338 and vice versa. Likewise, any description of the non-volatile memory 216 and volatile memory 220 may apply to the non-volatile and volatile memory of the computing device 338 and vice versa. Similarly, any description of the output device 208 may apply to the output of the computing device 338 and vice versa.

The computing device 338 may include a network interface for connecting to a fertility tracking engine 312 via a network 336. The fertility tracking engine 312 comprises volatile and non-volatile memory for storing fertility data associated with a unique identifier for identifying the user associated with the computing device 338. The fertility tracking engine 312 further includes a processor for executing programming instructions in the form of applications. Any description of the processor 204 may apply to the processor of the fertility tracking engine 312 and vice versa. Likewise, any description of the non-volatile memory 216 and volatile memory 220 may apply to the non-volatile and volatile memory of the fertility tracking engine 312 and vice versa.

Reference data may be stored in memory at the microcontroller 116, computing device 338, or fertility tracking engine 312. The reference data comprises feedback signals obtained from a plurality of test subjects using the sensor unit 101. The reference data may be associated with temporal, physiological, and demographic data. Temporal data may comprise a phase or a day within a reproductive cycle. Phases of the reproductive cycle include luteal phase, follicular phase, ovulation, fertile phase, proliferative phase, secretory phase, period, pregnancy, and the like. A day may be indicated as “Day 1 of 31” or the like. Physiological data may comprise a physiological indicator corresponding with the respective temporal data. In a specific example, the physiological data may comprise average body temperatures corresponding to days of the reproductive cycle. The demographic data may include age, weight, ethnicity, health status, disease state, and the like. The reference data may represent feedback data obtained for known biological fluids, including recorded times, electrical properties of the feedback signal, the number and placement of active conductive threads, and the gap distance between the conductive threads. A person of skill in the art will understand that the reference data may represent the average human reproductive cycle and the biological liquids secreted during respective phases of the human reproductive cycle.

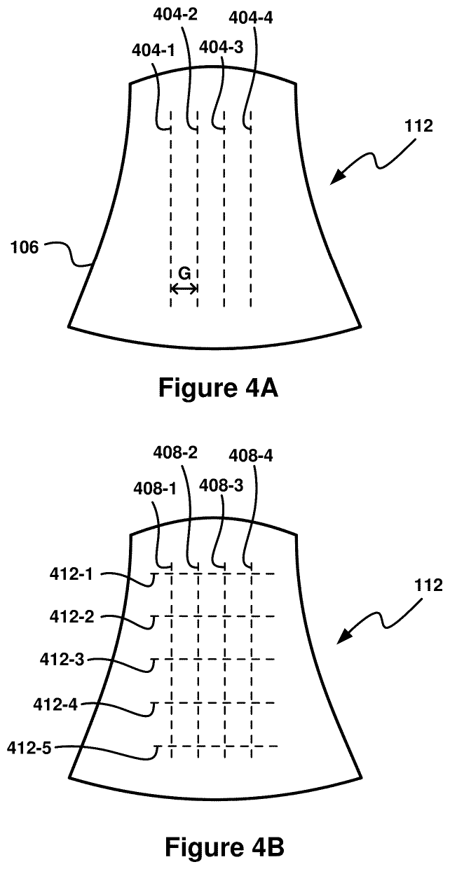

FIG. 4A is a top elevation view of an example sensing element 112-1 from FIG. 1. In the embodiment shown in FIG. 4A, the sensing element 112-1 is incorporated into the gusset 106 of the wearable device 100, however the sensing element 112-1 may be incorporated into any suitable textile.

The sensing element 112-1 comprises a plurality of conductive threads 404-1, 404-2, 404-3, 404-4 (referred to herein generally as “conductive thread 404” or collectively as “conductive threads 304”) disposed on the textile. The sensing element 112-1 may include any suitable number of conductive threads 404. In some examples, the sensing element 112-1 includes two conductive threads 404. In some examples, the sensing element 112-1 includes four conductive threads 404. In some examples, the sensing element 112-1 includes six conductive threads 404. In some examples, the sensing element 112-1 includes ten conductive threads 404. In some examples, the sensing element 112-1 includes twenty conductive threads 404. In some examples, the sensing element 112-1 includes one hundred conductive threads 404. In examples where the textile comprises a woven fabric, the conductive threads 404 may be integrated into the textile during the weaving of the textile. In other examples, the conductive threads 404 are stitched onto the textile.

In specific, non-limiting examples, the conductive threads 404 comprise stainless steel yarn. In other non-limiting examples, the conductive threads 404 comprise a thread with a conductive coating. The conductive thread may comprise any suitable natural or synthetic fiber. The conductive coating may comprise a conductive metal such as copper, gold, silver, or the like. The conductive coating may comprise a carbon-based nanostructure such as carbon nanotubes or graphene. In particular examples, the conductive threads 404 comprise silver-coated threads. The conductive threads 404 may be selected for stability, washability, ability to dry quickly, repeatability, durability, flexibility, biocompatibility, and antimicrobial properties. Specific non-limiting examples of conductive threads can be obtained from Mayata™, Shieldex™, VtechTextile™, Seeed Studio™, and other suitable suppliers.

The conductive threads 404 are spaced apart in the textile. In some examples, the conductive threads 404 are equidistant or approximately equidistant. In other examples, the gap distance G between adjacent conductive threads 404 varies, with some of the conductive threads 404 positioned closer and others positioned farther apart. The gap distance G between adjacent conductive threads 404 may be selected based on the desired sensitivity or properties of the biological liquid. The gap distance G between adjacent conductive threads may range from about 0.5 mm to about 100 mm. In specific examples, the gap distance G is about 0.5 mm. In specific examples, the gap distance G is about 1 mm. In specific examples, the gap distance G is about 2 mm. In specific examples, the gap distance G is about 4 mm. In specific examples, the gap distance G is about 6 mm. In specific examples, the gap distance G is about 8 mm. In specific examples, the gap distance G is about 10 mm. In specific examples, the gap distance G is about 15 mm. In specific examples, the gap distance G is about 10 mm. In specific examples, the gap distance G is about 20 mm.

It should be understood that the sensitivity of the sensor unit 101 is correlated with the number of the conductive threads 404 and the respective gap distances G. Generally, if the first and second conductive threads 404-1, 404-2 are positioned close together, the sensor unit 101 will be able to detect even small volumes of biological liquid. Furthermore, the precision will be correlated with the number of the conductive threads 404 included in the sensor unit 101.

Preferably, the conductive threads 404 are not in contact with each other, so that the conductive threads 404 are not electrically connected. Any number of configurations are contemplated for spacing the conductive threads 404. In FIG. 4A, the conductive threads 404 are spaced apart and parallel, however the conductive threads 404 are not particularly limited. In some embodiments, the conductive threads are aligned in straight lines, curved lines, zigzags, radial pattern, grid pattern, abstract shapes, or the like.

Another embodiment of the sensing element 112-2 is shown in FIG. 4B, which is a top elevation view of the sensing element 112-2. In FIG. 4B, the conductive threads 404 of the sensing element 112-2 are arranged in a grid pattern. The conductive threads 404 include a first set of parallel threads 408 and a second set of parallel threads 412. The first set of parallel threads 408-1, 408-2, 408-3, 308-4 (referred to herein generally as “first set of parallel threads 408”) are substantially perpendicular to the second set of parallel threads 412-1, 412-2, 412-3, 412-4, 412-5 (referred to herein generally as “second set of parallel threads 412”). Although the parallel threads are perpendicular, the first set of parallel threads 408 does not contact the second set of parallel threads 412. The sensing element 112-2 may improve the accuracy of liquid volumes assessed by the sensor unit 101 as opposed to the linear arrangement of threads in the sensing element 112-1, because the grid pattern is capable of detecting the spread of a liquid in two dimensions.

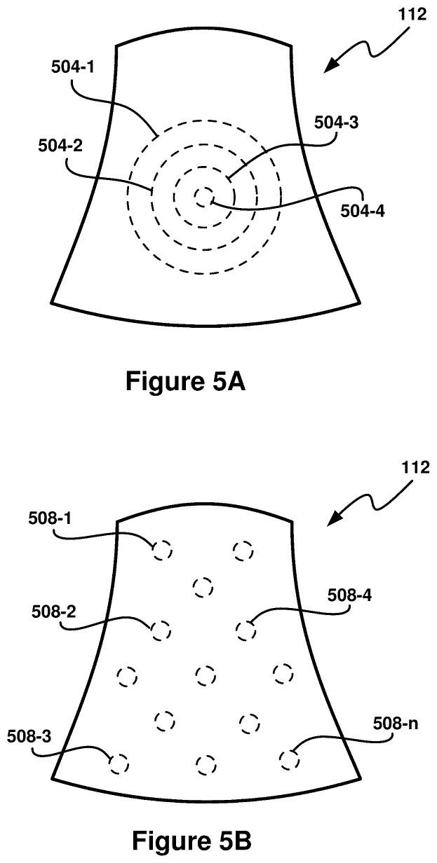

FIG. 5A shows another embodiment of the sensing element 112-3 in which the conductive threads 404 are arranged as a plurality of concentric circles. In FIG. 5A, the conductive threads 404 include a first concentric thread 504-1, a second concentric thread 504-2, a third concentric thread 504-3, and a fourth concentric thread 504-4, however the sensing element 112-3 may include any suitable number of concentric threads. In the embodiment shown in FIG. 5A, a single set of concentric circles is shown, however the sensing element 112-3 is not particularly limited. In other embodiments, the sensing element 112-3 may include multiple sets of concentric circles distributed over the textile.

FIG. 5B shows another embodiment of the sensing element 112-4 in which the conductive threads 404 are arranged as a plurality of scattered dots. In FIG. 5B, the conductive threads 404 include a first conductive thread 508-1, a second conductive thread 508-2, a third conductive thread 508-3, a fourth conductive thread 508-4, and an n^{th} conductive thread 508-n.

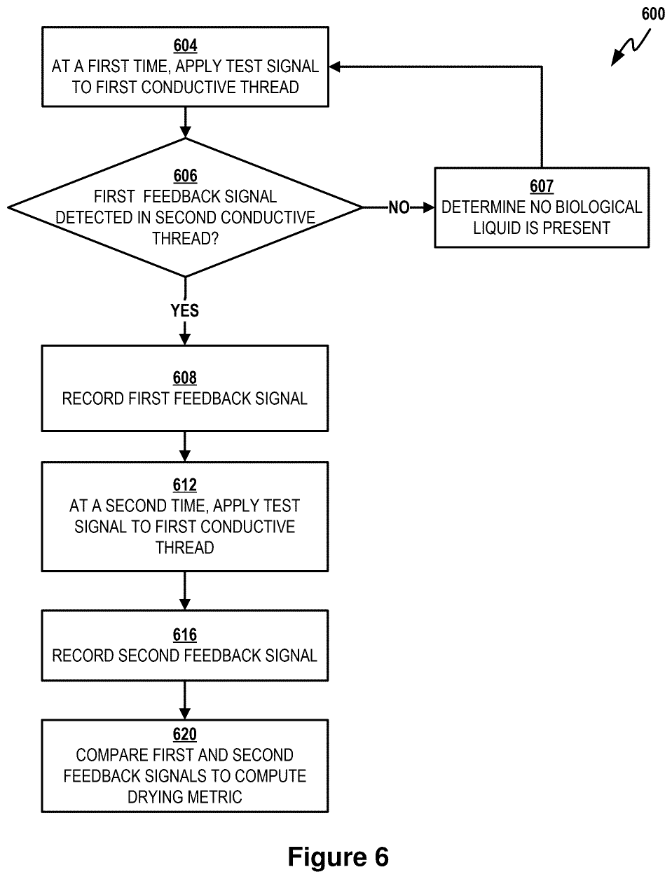

FIG. 6 is a block diagram of a method 600 for characterizing a biological liquid using the sensor unit 101 of FIG. 1, according to one embodiment. FIG. 6 will be explained with reference to the sensing element 112-1 of FIG. 3A, however FIG. 6 can similarly be applied to other iterations of the sensing element 112.

Block 604 comprises applying a test signal to a first conductive thread at a first time. In the sensor unit 101, block 604 is performed by the microcontroller 116 which applies a test signal to the first conductive thread 404. In the embodiment shown in FIG. 4A, the microcontroller 116 may apply the test signal to the first conductive thread 404-1. In some examples, block 604 comprises applying the test signal to a plurality of first conductive threads. In the embodiment shown in FIG. 4B, the microcontroller 116 may apply the test signal to a first one of the first set of parallel threads 408-1 and further apply the test signal to a first one of the second set of parallel threads 412-1. In a further, non-limiting example, the microcontroller 116 applies the test signal to threads 408-2 and 412-3. It should be understood that the order of the conductive threads 404 is not particularly limited to the arrangement shown in FIG. 4A or 4B, and in other examples, the first conductive thread 304-1 may be arranged between the second conductive thread 404-2 and the third conductive threads 404-3. Other arrangements are contemplated.

In examples where the microcontroller 116 applies the test signal to a plurality of first conductive threads 404, the microcontroller 116 may apply the test signal to alternating conductive threads 404. In other examples, the microcontroller 116 may apply the test signal to every third conductive thread 404. In other examples, the microcontroller 116 may apply the test signal to every fourth conductive thread 404. In the example sensing element 112 shown in FIG. 4A, the microcontroller 116 may apply the test signal to the second conductive thread 204-1 and the fourth conductive thread 204-3.

The test signal comprises an electrical current. The electrical current may have a current between about 0.01 mA and about 0.1 mA, although the current is not particularly limited.

Block 606 comprises determining whether a first feedback signal is detected in the second conductive thread. In the sensor unit 101, block 606 is performed by the microcontroller 116 which detects whether the first feedback signal has been detected in the second conductive thread 404-2, the first feedback signal responsive to the test signal.

If no biological liquid is deposited on the textile to connect the first conductive thread 304-1 and the second conductive thread 404-2, the microcontroller 116 determines that no biological liquid is present on the textile, as shown at block 607, and the method 600 returns to block 604. Generally, data recorded when no biological liquid is present at the first time is disregarded or relevant data records are deleted from memory. Block 604 may be repeated continuously or periodically. In examples where the test signal is applied periodically, the frequency at which the test signal is applied may be between about 1 second and about 60 minutes. In some examples, the test signal is applied every 10 seconds. In some examples, the test signal is applied every 30 seconds. In some examples, the test signal is applied every 60 seconds. In some examples, the test signal is applied every 2 minutes. In some examples, the test signal is applied every 10 minutes. In some examples, the test signal is applied every 20 minutes. In some examples, the test signal is applied every 30 minutes. In some examples, the test signal is applied every 40 minutes. In some examples, the test signal is applied every 50 minutes. In some examples, the test signal is applied every hour.

If the biological liquid is deposited on the textile such that the biological liquid contacts the first conductive thread 404-1 and the second conductive thread 404-2, forming a conductive bridge, therebetween, the microcontroller 116 will detect the first feedback signal in the second conductive thread 404-2, and the method 600 proceeds to block 608. The first feedback signal will be detected in the second conductive threads 404-2 as long as the biological liquid contacts both the first and second conductive threads 404-1, 404-2 somewhere along the respective lengths and electrically connects the two conductive threads 404-1, 404-2.

The biological liquid is not particularly limited and may include sebum, sweat, vaginal discharge, cervical mucus, urine, blood, amniotic fluid, lochia, or the like.

Block 608 comprises recording the first feedback signal. In the sensor unit 101, block 608 is performed by the microcontroller 116 which records in memory data representing the first feedback signal.

In some examples, block 608 includes measuring one or more electrical properties of the first feedback signal. The one or more electrical properties of the first feedback signal may be stored in memory. In preferred embodiments, the electrical property of the feedback signal is voltage since voltage is generally unaffected by contact between the biological liquid and the wearer's skin.

In some examples, block 608 includes detecting the first feedback signal in a plurality of second conductive threads and determining how many of the second conductive threads are conveying the first feedback signal. The number of second conductive threads may be stored in memory. Each of the conductive threads 404 may be associated with a unique identifier and as part of block 608, the microcontroller 116 may record the unique identifiers associated with each of the conductive threads 404 that convey the first feedback signal.

In some examples, block 608 includes recording the first time. In these examples, the microcontroller 116 includes a clock configured to record time. When the test signal is applied to the first conductive thread 404, the microcontroller 116 may retrieve the time from the clock and store the first time in memory. As part of this step, the microcontroller 116 may further retrieve the data and store the date in memory.

Block 612 comprises applying the test signal to the first conductive thread at a second time. In the sensor unit 101, block 612 is performed by the microcontroller 116 which re-applies a test signal to the first conductive thread 404 at a second time. The second time may be spaced from the first time by an interval. The interval may be between about 1 second and about 60 minutes. In some examples, the interval is about 10 seconds. In some examples, the interval is about 30 seconds. In some examples, the interval is about 60 seconds. In some examples, the interval is about 2 minutes. In some examples, the interval is about 10 minutes. In some examples, the interval is about 20 minutes. In some examples, the interval is about 30 minutes. In some examples, the interval is about 40 minutes. In some examples, the interval is about 50 minutes. In some examples, the interval is about 60 minutes.

Any description of block 604 generally applies to block 612.

Block 616 comprises recording the second feedback signal. In the sensor unit 101, block 616 is performed by the microcontroller 116 which records in memory data representing the second feedback signal. Any description of block 616 similarly applies to 616. Preferably, the microcontroller 116 measures and records corresponding data about the first and second feedback signals.

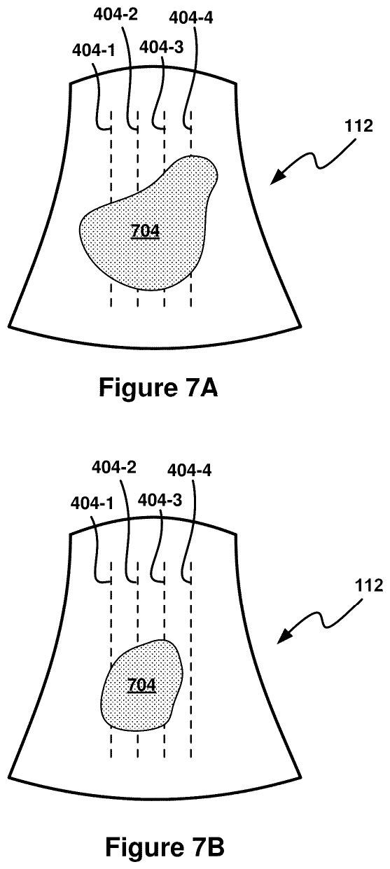

FIGS. 7A and 7B shows exemplary performance of blocks 604 to and 616 as performed by using the sensor unit 101 on which a biological liquid 704 has been deposited. FIG. 7A shows the biological liquid 704 at a first time when the test signal is applied to the first conductive thread 404-1. Since the biological liquid forms a bridge between all four of the conductive threads 404-1, 404-2, 404-3, 404-4, the first feedback signal is detected in the second, third and fourth conductive threads 404-2, 404-3, 404-4. After an interval, the test signal is reapplied to the first conductive thread 404-1, but since a portion of the biological liquid 704 has evaporated, the second feedback signal is only detected in the second and third conductive threads 404-2, 404-3. The microcontroller 116

The data recorded at blocks 616 and 606 about the first and second feedback signals may be stored in a database 228. Table 1 provides a specific, non-limiting example of a database storing properties of a first feedback signal obtained at a first time and a second feedback signal obtained at a second time. As shown in the first column, each of the feedback signals stored in Table 1 is associated with a unique identifier indicating one of the conductive threads 404. In the example provided in Table 1, the first time is 14:02 and the second time is 14:35. Table 1 also includes the voltage of the feedback signals.

TABLE 1

| Thread Identifier | Time | Voltage (V) |
|---|---|---|
| T2 | 14:02 | 0.31 |
| T3 | 14:02 | 0.27 |
| T4 | 14:02 | 0.13 |
| T2 | 14:35 | 0.24 |
| T3 | 14:35 | 0.14 |
| T4 | 14:35 | 0.00 |

Block 620 comprises comparing the first and second feedback signals to evaluate a drying behaviour of the biological liquid. In the sensor unit 101 of FIG. 1, block 620 may be performed by the microcontroller 116 which analyzes the first and second signals received from the sensing element 112. In the fertility monitoring system 300 of FIG. 3, block 620 may be performed by the computing device 338 or the fertility tracking engine 312, having received the data about the first and second feedback signals from the sensor unit 101 via the network interface 232.

Generally, block 620 comprises applying an algorithm to the first and second feedback signals to compute a drying metric for the biological liquid. In specific, non-limiting examples, the algorithm is based on machine learning, deep-learning, neural networks, the like, and combinations thereof, which are trained to improve the accuracy of the drying metric computed at block 620. The drying metric may include the drying time, drying rate, the like, or a combination thereof.

The one or more machine-learning algorithms and/or deep learning algorithms and/or neural networks of the applications 824 may include, but are not limited to: a generalized linear regression algorithm; a random forest algorithm; a support vector machine algorithm; a gradient boosting regression algorithm; a decision tree algorithm; a generalized additive model; neural network algorithms; deep learning algorithms; evolutionary programming algorithms; Bayesian inference algorithms; reinforcement learning algorithms, and the like. However, generalized linear regression algorithms, random forest algorithms, support vector machine algorithms, gradient boosting regression algorithms, decision tree algorithms, generalized additive models, and the like may be preferred over neural network algorithms, deep learning algorithms, evolutionary programming algorithms, and the like. However, generalized linear regression algorithms, random forest algorithms, support vector machine algorithms, gradient boosting regression algorithms, decision tree algorithms, generalized additive models, and the like may be preferred over neural network algorithms, deep learning algorithms, evolutionary programming algorithms, and the like. To be clear, any suitable machine-learning algorithm and/or deep learning algorithm and/or neural network is within the scope of present examples.

The comparison at block 620 includes computing the difference between the first and second times to obtain a time difference. The drying metric is generally computed based on the time difference, and one or more additional properties of the feedback signals such as an electrical property, the number and placement of active conductive threads, and the gap distance between the conductive threads 404.

In examples where the microcontroller 116 is configured to measure an electrical property of the first and second feedback signals, the comparison at block 620 may include determining the difference between the electrical properties the first and second feedback signals. In particular examples, block 620 includes computing a voltage difference between the voltage of the first and second feedback signals. Based on the time difference between the first and second feedback signals, and the voltage difference between the first and second feedback signals, the algorithm can compute the drying metric of the biological liquid.

In examples where the microcontroller 116 is configured to determine the number of the conductive threads from which the first and second feedback signals are recorded, the comparison at block 620 may include comparing the number of conductive threads from which the first and second feedback signals are recorded. Based on the time difference between the first and second times, and the change in the number of active conductive threads, the algorithm can compute the drying metric of the biological liquid.

In some examples, block 620 includes retrieving from memory the gap distance G between the first and second conductive threads 404-1, 404-2 and determining the drying metric for the biological liquid based on both the time difference and the gap distance. The gap distance G may be stored in memory at the microcontroller 116, the computing device 338, or the fertility tracking engine 312. The gap distance G is generally stored in association with unique identifiers for the first and second conductive threads 404. The gap distance is relevant to the analysis because the electrical resistance between two conductive threads 404 is influenced by the difference. Furthermore, the gap distance corresponds to the volume of liquid, with larger volumes of liquid drying slower, and faster volumes of liquid drying quicker.

As a further part of block 620, the microcontroller 116, computing device 338 or the fertility tracking engine 312 may characterize the biological liquid based on the drying metric. The characterization may be based on a comparison between the drying rate and the reference data, which may be retrieved from memory. The characterization may be further based on user-generated data input at the computing device 338. Based on similarities between the reference data and the drying metric, the characterization may include identifying the biological liquid. In specific, non-limiting examples, the biological liquid may be identified as sebum, sweat, vaginal discharge, cervical mucus, urine, blood, amniotic fluid, lochia, the like, or a combination thereof. The characterization may include computing the initial volume of the biological liquid that was deposited on the textile, based on the drying metric. Generally, a large volume of liquid will dry more slowly than a small volume of liquid.

As a further part of block 620, the method 600 may include identifying a reproductive status of the user. The reproductive status may represent a particular day or phase in the user's reproductive cycle. The reproductive status may be determined based on the characterization of the biological liquid, including the drying metric, volume, and identified type of liquid. The reproductive status may be further determined based on the reference data, the user-generated data, and other sensor data. In addition to characterizing the reproductive status, block 620 may comprise determining a disease condition such as endometriosis, uterine fibroids, gynecologic cancer, polycystic ovary syndrome, congenital adrenal hyperplasia, sexually transmitted diseases, and the like.

The reproductive status generated at block 620 may be output at a display associated with the computing device 338.

In view of the above, it will now be apparent that variant, combinations, and subsets of the foregoing embodiments are contemplated.

While the method 600 has been described above with respect to a first and second feedback signal recorded at a first and second time, it should be understood that any suitable number of feedback signals may be recorded. In some examples, the microcontroller 116 applies the test signal repeatedly to the first conductive thread at a plurality of times, including the first and second time, and records a plurality of feedback signals, including the first and second signal. In these examples, the test signal may be applied continuously or periodically to the first conductive thread. The microcontroller 116 may be configured to record an end time when the second conductive thread first ceases to transmit the corresponding feedback signals. The end time may be the earliest recorded time when the measured feedback signal in the second conductive thread is 0 V. Thus, the comparison at block 620 includes computing a feedback signal duration by subtracting the first time from the end time. The feedback signal duration may indicate either the rate of absorption for the liquid or the rate of evaporation, or a combination thereof. A larger volume of liquid deposited on the textile typically results in a longer duration of the feedback signal, and a small volume of liquid typically results in a shorter duration of the feedback signal.

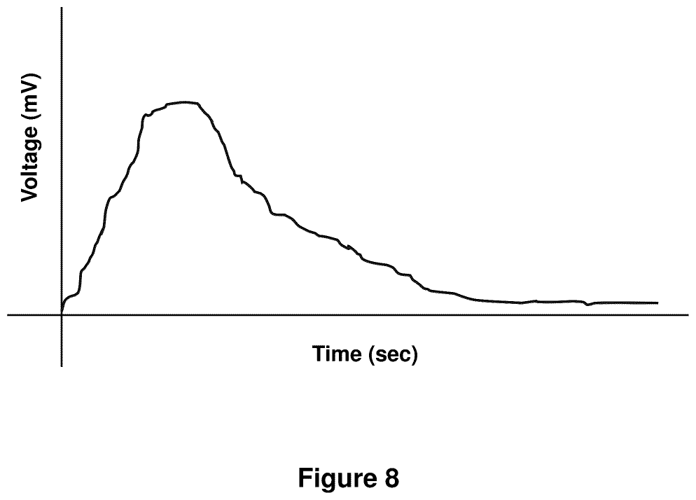

The voltage may change over time, and as part of method 600, the microcontroller 116 may measure the change in voltage over time. An example of the change in voltage over time is shown in FIG. 8. FIG. 8 is a graph where the voltage detected in the sensing element 112 is plotted on the y-axis, and time is plotted on the x-axis. When a liquid is added to the sensing element 112, the voltage initially increases rapidly until it reaches a peak. After peaking, the voltage gradually decreases as the liquid distributes and evaporates. The change in voltage over time may be used to uniquely identify the biological liquid by comparison to reference data for known biological liquids.

While the first and second feedback signals have been described above with respect to the second conductive thread 404-2, it should be understood that the feedback signals may be measured from any suitable number of conductive threads 404. In some examples, the microcontroller 116 includes one clock which records the feedback signal duration beginning when the feedback signal is first detected in one of the conductive threads 404, and ending when the feedback signal is no longer detected in any of the conductive threads 404. In other examples, the microcontroller 116 includes a plurality of clocks configured to time the feedback signal duration in the plurality of conductive threads 404.

In some examples, the microcontroller 116 includes a second clock for timing how long the feedback signal is at the highest voltage. When the voltage decreases or when the voltage decreases below a threshold, the microcontroller 116 stops the second clock and records the duration of maximum voltage. Generally, a maximum voltage is recorded when the sensing element 112 is saturated and, the voltage of the feedback signal decreases as the biological liquid dries. Larger volumes of liquid deposited on the textile generally result in longer durations of maximum voltage whereas smaller volumes of liquid generally result in shorter durations of maximum voltage. Therefore, the duration of maximum voltage can be correlated to the volume of the liquid.

In some instances, additional liquid is deposited onto the sensing element 112 before the feedback signal reaches 0 V. In these examples, the drying time will be lengthened by the addition of further liquid. Because the sensing element 112 is saturated a second time, the second clock may record a second duration of maximum voltage.

While the sensor unit 101 and the fertility monitoring system 300 were discussed above in relation to determining the reproductive status of a user, other health statuses are contemplated. In some examples, the sensor unit 101 and the fertility monitoring system 300 may be used for disease detection, fitness tracking, skin health, hydration monitoring, nutrition planning, stress detection, and the like. In these examples, the fertility tracking engine 312 may be a health tracking engine configured to determine the health status of the user.

It will now be apparent to a person of skill in the art that the present specification affords certain advantages over the prior art. Firstly, evaluating the drying behavior (rather than merely detecting moisture) enables physiological inferences to be drawn, such as the reproductive status of the user. Secondly, the yarn-based sensors are flexible, comfortable, and capable of seamless integration into a wearable textile without compromising garment form or function. Thirdly, the specific position of the conductive yarns is not particularly limited, improving robustness and reliability during use.

In view of the above, it will now be apparent that variant, combinations, and subsets of the foregoing embodiments are contemplated. For example, while the wearable device has been described with respective to fertility monitoring, a skilled person will understand that the device and method can be similarly applied to other applications such as cancer detection, monitoring and detecting infectious diseases such as bacterial vaginosis, menopause monitoring, fitness monitoring, wellness, athletic training and performance, sleep tracking, and the like.

It will now be apparent to a person of skill in the art that the present specification affords certain advantages over the prior art.

Moisture monitoring systems identify the presence of moisture and notify the user when a leak occurs. In contrast, the sensor unit described herein assesses a specific attribute of the biological liquid, offering valuable insights about rate and duration of drying. Such details are beneficial for advanced applications, including fertility tracking, as they allow for a deeper understanding of the properties of the biological liquid and enable users to make informed deductions based on those attributes.

The many features and advantages of the invention are apparent from the detailed specification and, thus, it is intended by the appended claims to cover all such features and advantages of the invention that fall within the true spirit and scope of the invention. Further, since numerous modifications and changes will readily occur to those skilled in the art, it is not desired to limit the invention to the exact construction and operation illustrated and described, and accordingly all suitable modifications and equivalents may be resorted to, falling within the scope of the invention.

What is claimed is:

1. A sensor unit for characterizing biological liquids, the sensor unit comprising:
a plurality of conductive threads incorporated into a textile, including at least a first conductive thread and a second conductive thread spaced from the first conductive thread;
a microcontroller electrically connected to the plurality of conductive threads, the microcontroller configured to:
apply a test signal to the first conductive thread at a plurality of times, including a first and second time,
record a plurality of feedback signals in at least the second conductive thread responsive to a biological liquid electrically connecting the first and second conductive threads, the plurality of feedback signals including at least a first and second feedback signal, corresponding to the first and second times, respectively;
compare the plurality of feedback signals; and
determine a drying metric for the biological liquid based on the comparison of feedback signals.

2. The sensor unit of claim 1, wherein the microcontroller is further configured to:
detect whether the first feedback signal is transmitted by the second conductive thread; and
if the first feedback signal is not transmitted, determine that no biological liquid is present on the textile.

3. The sensor unit of claim 1, wherein the microcontroller is further configured to:
retrieve from memory a gap distance between the first and second conductive threads; and
determine the drying metric for the biological liquid based on the gap distance.

4. The sensor unit of claim 1 wherein the microcontroller is further configured to determine a number of the conductive threads from which the first and second feedback signals are recorded, and wherein the comparison of feedback signals includes comparing the respective number of conductive threads from which the first and second feedback signals are recorded.

5. The sensor unit of claim 1 wherein the microcontroller is further configured to measure the voltage of the first and second feedback signals, and wherein the comparison of feedback signals includes determining a difference between the voltage of the first and second feedback signals.

6. The sensor unit of claim 1 wherein the microcontroller is further configured to record the plurality of times, and wherein the comparison of feedback signals includes determining a time difference between the first and second times.

7. The sensor unit of claim 1 wherein
the microcontroller is further configured to record an end time when the second conductive thread ceases to transmit the feedback signals; and
the comparison of feedback signals includes computing a feedback signal duration based on the first time and the end time.

8. The sensor unit of claim 7 wherein
the microcontroller is further configured to measure the voltage of the feedback signals; and
the comparison of feedback signals includes computing a rate of change in the voltage.

9. The sensor unit of claim 1:
wherein the plurality of conductive threads is arranged in a grid pattern on the textile, the plurality of conductive threads comprising:
a first set of parallel threads; and
a second set of parallel threads perpendicular to the first set of parallel threads; and
wherein the microcontroller is further configured to apply the test signal to a first one of the first set of parallel threads and a first one of the second set of parallel threads.

10. A wearable device comprising:
a garment comprising a textile configured to be worn by a user; and
the sensor unit according to claim 1.

11. A fertility monitoring system comprising:
a sensor unit for detecting a biological liquid, the sensor unit including:
a plurality of conductive threads incorporated into a textile, including at least
a first conductive thread and a second conductive thread spaced from the first conductive thread;
a microcontroller electrically connected to the plurality of conductive threads, the microcontroller configured to:
apply a test signal to the first conductive thread at a plurality of times, including a first and second time,
record a plurality of feedback signals in at least the second conductive thread responsive to the biological liquid electrically connecting the first and second conductive threads, the plurality of feedback signals including at least a first and second feedback signal, corresponding to the first and second times, respectively;
transmit the plurality of feedback signals; and
a computing device configured to:
receive the plurality of feedback signals from the sensor unit;
compare the plurality of feedback signals; and
determine a drying metric for the biological liquid based on the comparison of feedback signals.

12. The fertility monitoring system of claim 11, wherein the microcontroller is further configured to:
detect whether the first feedback signal is transmitted by the second conductive thread; and
if the first feedback signal is not transmitted, determine that no biological liquid is present on the textile.

13. The fertility monitoring system of claim 11, wherein the computing device is further configured to:
retrieve from memory a gap distance between the first and second conductive threads; and
determine the drying metric for the biological liquid based on the gap distance.

14. The fertility monitoring system of claim 11,
wherein the microcontroller is further configured to determine a number of the conductive threads from which the first and second feedback signals are recorded, and transmit the respective number of conductive threads to the computing device; and
wherein the comparison includes comparing the respective number of conductive threads.

15. The fertility monitoring system of claim 11
wherein the microcontroller is further configured to measure the voltage of the first and second feedback signals and transmit the respective voltages to the computing device; and
wherein the comparison includes determining a difference between the respective voltages.

16. The fertility monitoring system of claim 11
wherein the microcontroller is further configured to record the first and second times and transmit the first and second times to the computing device; and
wherein the comparison of feedback signals includes determining a time difference between the first and second times.

17. The fertility monitoring system of claim 11,
wherein the microcontroller is further configured to record an end time when the microcontroller ceases to receive the feedback signal, and transmit the end time to the computing device; and
wherein the comparison of feedback signals includes computing a feedback signal duration based on the first time and the end time.

18. The fertility monitoring system of claim 17,
wherein the microcontroller is further configured to measure the voltage of the corresponding feedback signals and transmit the voltage of the feedback signals to the computing device; and
wherein the comparison includes computing a rate of change in the voltage of the feedback signals.

19. The fertility monitoring system of claim 11:
wherein the plurality of conductive threads is arranged in a grid pattern on the textile, the plurality of conductive threads comprising:
a first set of parallel threads; and
a second set of parallel threads perpendicular to the first set of parallel threads; and
wherein the microcontroller is further configured to apply the test signal to a first one of the first set of parallel threads and a first one of the second set of parallel threads.

20. The fertility monitoring system of claim 11 wherein the computing device is further configured to:
compare the drying metric of the biological liquid to reference data; and
determine a reproductive status of a user based on the comparison between the drying metric and the reference data.
